(19) United States
(12) Patent Application Publication
(10) Pub. No.: US 2020/0305376 A1
(43) Pub. Date: Oct. 1, 2020

(54) POWDERY MILDEW RESISTANT SUNFLOWER

(71) Applicant: SYNGENTA PARTICIPATIONS AG, Basel (CH)

(72) Inventor: Shifeng PAN, Gilroy, CA (US)

(73) Assignee: SYNGENTA PARTICIPATIONS AG, Basel (CH)

(21) Appl. No.: 16/954,678

(22) PCT Filed: Feb. 8, 2019
(86) PCT No.: PCT/US2019/017164

§ 371 Date: Jun. 17, 2020

Related U.S. Application Data
(60) Provisional application No. 62/629,813, filed on Feb. 13, 2018.

(51) Int. Cl. A01H 6/14; A01H 5/10

(52) U.S. Cl. CPC A01H 6/1464; A01H 5/10

(57) ABSTRACT

Helianthus annuus are susceptible to powdery mildew caused by fungi in the order Erysiphales. The present invention relates to novel Helianthus interspecific hybrids containing a genetic resistance to powdery mildew. The present invention also relates to methods for creating novel Helianthus interspecific hybrids containing a genetic resistance to powdery mildew and Helianthus interspecific hybrids produced by the method.

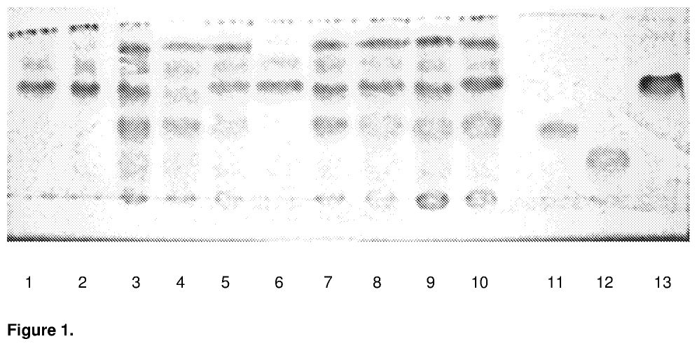

This application claims priority under 35 U.S.C. § 119 to U.S. Provisional Application Ser. No. 62/629,813, filed Feb. 13, 2018, which is incorporated herein in its entirety by reference.

### INTRODUCTION

Powdery Mildew caused by fungi in the order Erysiphales first appears on infected plants with the symptoms of a white to gray coating on the upper leaf surface. Powdery mildew is common on ornamental sunflowers grown in greenhouses and can result in plants which are not saleable. In the field, powdery mildew is seen late in the season. Infected areas of the plant senesce and dry up. The novel interspecific hybrid sunflower made from H. annuus x H. argophyllus x H. debilis is resistant to Powdery mildew providing a solution for ornamental sunflower growers.

Helianthus is a genus of plants comprising about 70 species in the family Asteraceae, all of which are native to North America except three species in South America. The common name “sunflower” also applies to the popular annual species Helianthus annuus, the common sunflower. This and other species, notably Jerusalem artichoke (H. tuberosus), are cultivated in temperate regions as food crops and ornamental plants.

The pot type sunflower market is rapidly growing in North America and the rest of the world. However, the current pot sunflowers on the market suffer from several disadvantages. They are often too tall to easily fit on racks for shipment, are not well-branched and have a short flowering time as well as being susceptible to Powdery mildew.

The present application describes a new indeterminate interspecific plant species made from Helianthus annuus x Helianthus argophyllus x Helianthus debilis plant which unexpectedly addresses these disadvantages. Unlike conventional Helianthus annuus plants, the plant of the present invention is resistant to Powdery mildew.

This new Powdery mildew resistant species has the potential to reduce losses to powdery mildew.

### SUMMARY OF THE INVENTION

The inventors of the present invention have developed an interspecific hybrid sunflower plant which has powdery mildew resistance as well as high levels of branching, high levels of continuous flowering and a mounding, spreading growth habit.

This new interspecific hybrid will provide a 10 plus week's flower time in garden comparing to 2 weeks flower time for traditional pot or cut flower varieties on the market today.

The present invention relates to an interspecific Helianthus plant comprising a genetic determinant responsible for resistance to Powdery mildew. This is a very novel multi-branch, multi-flower, and mound spreading plant. The plant will continue flower in a garden for 10 plus weeks. In some embodiments, the Powdery mildew resistant interspecific Helianthus plant is resistant to Erysiphe cichoracearum. In other embodiments, the Powdery mildew resistant interspecific Helianthus plant is resistant to Podosphaera xanthii.

In one embodiment, the interspecific Helianthus plant of the invention is obtainable by crossing with a Helianthus plant comprising a genetic determinant responsible for resistance to Powdery mildew, wherein a sample of representative seed of said plant comprising a genetic determinant for resistance to Powdery mildew, Helianthus Interspecific Hybrid SUR67-1 (4A994), is deposited under ATTC Accession No. PTA-125012.

The present invention provides a Helianthus plant comprising a genetic determinant responsible for resistance to Powdery mildew, wherein said genetic determinant responsible for resistance to Powdery mildew can be found in a plant grown from seed deposited under ATTC Accession No. PTA-125012.

The present invention also provides Helianthus seed comprising a genetic determinant responsible for resistance to Powdery mildew, wherein said seed is obtainable from a plant grown from seed deposited under ATTC Accession No. PTA-125012, or progeny thereof.

The present invention provides Helianthus seed from a plant of the present invention.

The present invention provides a Helianthus plant produced by growing seed from a plant of the present invention. In some embodiments, the Helianthus plant is an ornamental plant.

In one embodiment, the plant of the invention is an interspecific plant obtainable from H. annuus, H. argophyllus, and H. debilis.

The present invention provides a method of producing hybrid Helianthus seed of a plant of the invention comprising crossing a first parent Helianthus plant with a second parent Helianthus plant and harvesting the resultant Fi hybrid Helianthus seed.

In one embodiment, the first Helianthus plant is H. annuus x H. argophyllus and the second Helianthus plant is H. debilis.

The present invention provides a seed of a plant produced by the method of the present invention.

The present invention provides a hybrid plant or its parts produced by growing said hybrid seed of the present invention. The present invention provides seed comprising a genetic determinant responsible for resistance to Powdery mildew produced from the hybrid plant of the present invention. The present invention provides viable Helianthus seed deposited under ATCC Accession No. PTA-125012 and plants grown from said deposited seed and the progeny thereof.

### DETAILED DESCRIPTION OF THE INVENTION

The present invention provides a Helianthus plant comprising a genetic determinant responsible for an indeterminate phenotype.

In one embodiment the Helianthus plant of the invention is interspecific, obtainable by crossing a Helianthus plant with another Helianthus plant comprising a genetic determinant responsible for resistance to Powdery mildew, wherein a sample of representative seed of said plant comprising a genetic determinant responsible for resistance to Powdery mildew is deposited under ATTC Accession No. PTA-125012. In one embodiment, the resistance to Powdery mildew is dominant.

In one embodiment, the plant of the invention is an interspecific plant made from H. annuus x H. argophyllus and H. debilis.

In one embodiment, the plant of the invention is an interspecific plant made from Helianthus debilis and another Helianthus plant.

In one embodiment, the plant height, number of flowers, plant width, and flower diameter is measured under typical environmental conditions as described herein. In one embodiment, these measurements are taken from the finished plant.

The plant of the invention is well branched and early to bloom as defined herein.

In one embodiment, the Helianthus plant according to the present invention is an annual plant.

The present invention provides a Helianthus plant comprising a genetic determinant responsible for resistance to Powdery mildew, wherein said genetic determinant responsible for resistance to Powdery mildew can be found in a plant grown from seed deposited under ATTC Accession No. PTA-125012.

The present invention provides Helianthus seed comprising a genetic determinant responsible for resistance to Powdery mildew, wherein said seed is obtainable from a plant grown from seed deposited under ATTC Accession No. PTA-125012, or progeny thereof. The present invention provides Helianthus seed comprising a genetic determinant responsible for resistance to Powdery mildew, wherein said seed has a pedigree which includes the plant Helianthus Interspecific Hybrid SUR67-1 (4A994), and wherein representative seed of said plant has been deposited under ATTC Accession No. PTA-125012. The present invention provides a Helianthus seed comprising a genetic determinant responsible for an indeterminate phenotype, wherein said genetic determinant is present in Helianthus Interspecific Hybrid SUR67-1 (4A994), a representative sample of seed which is deposited at ATTC under Accession No. ATTC PTA-125012, or in a progeny or ancestor thereof comprising said genetic determinant responsible for resistance to Powdery mildew.

In one embodiment, the plant of the invention is an elite Helianthus plant comprising a genetic determinant responsible for resistance to Powdery mildew. In one embodiment, Helianthus seed from a plant of the invention comprises a genetic determinant responsible for resistance to Powdery mildew.

The present invention provides a Helianthus plant produced by growing the seed of the present invention. In one embodiment, the Helianthus plant of the invention is a hybrid. In one embodiment, the hybrid Helianthus plant of the invention is male sterile.

The present invention provides a tissue culture of cells produced from a Helianthus plant of the present invention, wherein said cells of the tissue culture are produced from a plant part selected from the group consisting of seed, leaf, pollen, embryo, cotyledon, hypocotyl, meristematic cell, root, root tip, pistil, anther, flower, stem, and petiole.

The present invention provides a Helianthus plant of the present invention regenerated from tissue culture.

The present invention provides an ovule of the Helianthus plant of the present invention.

The present invention provides a method of producing hybrid Helianthus seed comprising crossing a first parent Helianthus plant with a second parent Helianthus plant and harvesting the resultant hybrid Helianthus seed.

In one embodiment, the first parent plant is an Interspecific Hybrid Helianthus annuus x Helianthus argophyllus comprising a genetic element responsible for an indeterminate phenotype. In one embodiment, the Helianthus debilis with a genetic determinant responsible for resistance to Powdery mildew is the female parent

The present invention further relates to a method of producing the said Helianthus plant and seed by crossing a plant of the instant invention with another Helianthus plant. The invention also relates to the transfer or introgression of the genetic determinant into genera other than Helianthus.

In order to transfer the heritable genetic determinant to another plant, backcross breeding can be used. For this a desirable homozygous cultivar or inbred is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent. A suitable donor parent for the genetic determinant for resistance to Powdery mildew according to the present invention may be the Helianthus plant Helianthus Interspecific Hybrid SUR67-1 (4A994), representative seeds of which have been deposited at American Type Culture Collection (ATCC) Patent Depository, 10801 University Blvd., Manassas, Va. 20110. The ATCC number of the deposit is PTA-PTA-125012. Accordingly, the person skilled in the art of Helianthus breeding, based on the description of the present invention and in possession of Helianthus plant Helianthus Interspecific Hybrid SUR67-1 (4A994), representative seed of which is deposited under accession Number PTA-125012, has no difficulty transferring the genetic determinant responsible for resistance to Powdery mildew according to the present invention to any other Helianthus plant using any breeding technique well known in the art.

The present invention provides hybrid seed produced by the method of the present invention.

The present invention provides a plant of the invention or its parts, including cut flowers, produced by growing seed of the present invention. In one embodiment, the plant of the invention is used in combination mix containers.

In one embodiment, the plant of the invention can be propagated by vegetative means.

The present invention provides seed comprising a genetic determinant responsible for resistance to Powdery mildew produced from the hybrid plant of the present invention. The present invention provides viable Helianthus seeds deposited under ATCC Accession No. PTA-125012 and plants grown from said deposited seeds and the progeny thereof, wherein the progeny contain the genetic determinant for resistance to Powdery mildew.

### Definitions

The technical terms and expressions used within the scope of this application are generally to be given the meaning commonly applied to them in the pertinent art of plant breeding and cultivation if not otherwise indicated herein below.

As used in this specification and the appended claims, the singular forms “a”, “an”, and “the” include plural referents unless the context clearly dictates otherwise. Thus, for example, reference to “a plant” includes one or more plants. As used herein, the term “breeding”, and grammatical variants thereof, refer to any process that generates a progeny individual. Breeding can be sexual or asexual, or any combination thereof. Exemplary non-limiting types of breeding include crossing, selfing, doubled haploid derivative generation, polyploidization and combinations thereof. The phenotype of the flower of the present invention can be readily and stably transferred by breeding to progeny.

As used herein, the term “hybrid” in the context of plant breeding refers to a plant that is the offspring of genetically dissimilar parents produced by crossing plants of different lines or breeds or species, including but not limited to the cross between two inbred lines.

A “cultivated Helianthus plant” or an “elite Helianthus plant” is understood within the scope of the invention to refer to a plant that is no longer in the natural state but has been developed by human care and for human use and/or consumption. An example of a cultivated plant or an elite plant is Helianthus Interspecific SUR67-1 (4A994), representative seed of which is deposited under accession Number PTA-125012. A “finished plant” is defined as a plant which has just started flowering. Typically, flowering starts at around 85 to 90 days from sowing (12 to 13 weeks) when grown under typical environmental conditions as described herein.

A “well branched plant” is defined as a plant which has 6 to 8 branches per plant in the pot at flowering time. Conventional pot type Helianthus plants only have 1 major stem per plant in pot.

An early to bloom plant is defined as a plant which blooms in about 85 to 90 days, when grown under typical environmental conditions as described herein. Typical environmental conditions for growing a plant of the invention are 14 to 16 hours of daylight and average daily temperature of 27 to 29 degrees Celsius, from 21 days after sowing in early spring to late fall in California. Unless otherwise stated, a plant of the invention displays the stated phenotype when grown under these typical environmental conditions.

An indeterminate plant is defined as a plant which is continually branching from when the first flower appears, continually has new shoots appearing, and is continually flowering when grown under the typical environmental conditions as described herein.

As used herein, the phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, callus, and the like. As used herein, the term “progeny” refers to the descendant(s) of a particular cross. Typically, progeny result from breeding of two individuals, although some species (particularly some plants and hermaphroditic animals) can be selfed (i.e. the same plant acts as the donor of both male and female gametes). The descendant(s) can be, for example, of the F1, the F₂, or any subsequent generation. “Trait” is understood within the scope of the invention to refer to a characteristic or phenotype. A trait may be inherited in a dominant or recessive manner, and may be monogenic or polygenic. “Dominant” is understood within the scope of the invention to refer to an allele which determines the phenotype when present in the heterozygous or homozygous state.

A “recessive” allele is only displayed when present in the homozygous state.

### Seed Deposit Details

Seed of the variety Helianthus Interspecific Hybrid SUR67-1 (4A994) made from H. annuus x H. argophyllus x H. debilis is deposited under the terms of the Budapest Treaty American Type Culture Collection (ATCC) Patent Depository, 10801 University Blvd., Manassas, Va. 20110. The ATCC number of the deposit is PTA-125012. The seed deposit is made in the name of Syngenta Participations AG, Basel 4002, Switzerland.

### EXAMPLES

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### Example 1

The Interspecific hybrid H. annuus x H. argophyllus named HELAC0001 is the female parent crossed with the male parent of the species H. debilis named HELAB0103-2V to produce the Interspecific hybrid named SUR67-1.

### Example 2

### Powdery Mildew Resistance

HELAC0001, HELAB0103-2V, and SUR67-1 where grown in Gilroy Calif., Vero Beach Fla., Andijk Netherlands, and Charlotte N.C. in summer 2017 under natural disease pressure and scored for Powdery mildew.

TABLE 1

| | Flower | Width | Height | Powdery Mildew |
|---|---|---|---|---|
| Line | Diameter(cm) | (cm) | (cm) | (0-5) |

2017Gilroy Field

| | | | | |
|---|---|---|---|---|
| HELAC0001 | 12.33 | 155 | 147 | 2 |
| SUR67-1 | 8.5 | 180 | 132 | 0 |
| HELAB0103-2V | 3 | 72 | 29 | 0 |

2017 Vero Beach Field

| | | | | |
|---|---|---|---|---|
| HELAC0001 | 11 | 114 | 117 | 2 |
| SUR67-1 | 8 | 91 | 114 | 0 |
| HELAB0103-2V | 3 | 50 | 27 | 0 |

2017 NL(Andijk) Field

| | | | | |
|---|---|---|---|---|
| HELAC0001 | 13 | 160 | 120 | 1 |
| SUR67-1 | 8 | 140 | 90 | 0 |
| HELAB0103-2V | 4 | 33 | 70 | 0 |

2017 Metrolina, NA Field

| | | | | |
|---|---|---|---|---|
| HELAC0001 | 15.2 | 101.6 | 127 | 2 |
| SUR67-1 | 8.9 | 139.7 | 96.5 | 0 |
| HELAB0103-2V | 4.2 | 53.3 | 33 | 0 |

Note:
HELAC0001 - Sunfinity sterile hybrid, female for vegetative SUR67-1
SUR67-1: Vegetative
HELAB0103-2V: Male parent for SUR67-1
0 => no symptoms
1 => just starting with PM on the stem next to the top leaf or flower stem.
2 => leaf has also a little PM damage.
3 => ½ of the plant affected with PM.
4 => almost the whole plant affected with PM.
5 => the whole plant affected with PM.

1. An interspecific Helianthus plant comprising a genetic determinant responsible for resistance to powdery mildew.

2. The interspecific Helianthus plant of claim 1, obtainable by crossing a Helianthus plant with another Helianthus plant comprising a genetic determinant responsible for resistance to Powdery mildew, wherein a sample of representative seed of said plant comprising a genetic determinant responsible for resistance to powdery mildew is deposited under ATCC Accession No. PTA-125012.

3. The interspecific Helianthus plant of claim 1, wherein said genetic determinant responsible for resistance to powdery mildew can be found in a plant grown from seed deposited under ATCC Accession No. PTA-125012.

4. The interspecific Helianthus plant of claim 1 wherein said plant is made from H. annuus x H. argophyllus x H. debilis.

5. Helianthus seed comprising a genetic determinant responsible for resistance to powdery mildew, wherein said seed is obtainable from a plant according to claim 1.

6. A Helianthus plant produced by growing the seed of claim 5.

7. A method of producing hybrid Helianthus seed according to claim 5 comprising crossing a first parent Helianthus plant with a second parent Helianthus plant and harvesting the resultant hybrid Helianthus seed.

8. Hybrid seed produced by the method of claim 7.

9. A hybrid plant or its parts produced by growing the hybrid seed of claim 8.

10. Seed comprising a genetic determinant responsible for resistance to powdery mildew produced from the hybrid plant of claim 9.
